(19) United States
(12) Patent Application Publication
(10) Pub. No.: US 2023/0016575 A1
(43) Pub. Date: Jan. 19, 2023

(54) ALTERNATING TANGENTIAL FLOW BIOREACTOR WITH HOLLOW FIBER SYSTEM AND METHOD OF USE

(71) Applicant: Repligen Corporation, Waltham, MA (US)

(72) Inventors: Bao Le, Redondo Beach, CA (US); Travis R. Ward, Waltham, MA (US)

(73) Assignee: Repligen Corporation, Waltham, MA (US)

(21) Appl. No.: 17/781,791

(22) PCT Filed: Dec. 14, 2020
(86) PCT No.: PCT/US20/64887

§ 371 Date: Jun. 2, 2022

Related U.S. Application Data
(60) Provisional application No. 62/947,989, filed on Dec. 13, 2019.

(51) Int. Cl. C12M 1/00; C12M 1/12; C12M 1/34

(52) U.S. Cl. CPC C12M 29/16; C12M 23/26; C12M 23/28; C12M 25/02; C12M 29/10; C12M 41/44; C12M 29/04

(57) ABSTRACT

Embodiments of the present disclosure relate generally to systems and methods for perfusion cell culture involving alternating fluid flows between first and second flexible vessels. For example, a hollow fiber filter module may be attached to first and second culture vessels which each include inner and outer vessels. A pressure source may cause a pressure differential between the outer vessels, which may cause a responsive fluid flow between the inner vessels across a hollow fiber filtration unit.

### CROSS REFERENCE TO RELATED APPLICATIONS

This application claims the benefit of priority under 35 U.S.C. § 119 to U.S. Provisional Patent Application Ser. No. 62/947,989, filed on Dec. 13, 2019, which is incorporated by reference in its entirety for all purposes.

### FIELD OF DISCLOSURE

This disclosure relates generally to process filtration systems, and more particularly to systems utilizing alternating tangential flow bioreactors.

### BACKGROUND

Filtration is typically performed to separate, clarify, modify and/or concentrate a fluid solution, mixture or suspension. In the biotechnology and pharmaceutical industries, filtration is vital for the successful production, processing, and testing of new drugs, diagnostics and other biological products. For example, in the process of manufacturing biologicals, using animal or microbial cell culture, filtration is done for clarification, selective removal and concentration of certain constituents from the culture media or to modify the media prior to further processing. Filtration may also be used to enhance productivity by maintaining a culture in perfusion at high cell concentration.

Biologics manufacturing processes have advanced through substantial process intensification. Both eukaryotic and microbial cell culture to produce recombinant proteins, virus-like particles (VLP), gene therapy particles, and vaccines now include cell growth techniques that can achieve 100e6 cells/ml or higher. This is achieved using cell retention devices that remove metabolic waste products and refresh the culture with additional nutrients. One of the most common means of cell retention is to perfuse a bioreactor culture using hollow fiber filtration using alternating tangential flow (ATF). Both commercial and development scale processes use a device that controls a diaphragm pump to perform ATF through a hollow fiber filter (see, e.g., U.S. Pat. No. 6,544,424) in which the pump and filter are encased in stainless steel and autoclaved prior to use in order to maintain sterility. For economy and flexibility many production facilities are striving to use disposable products, however the conversion of the stainless steel ATF to a disposable pre-sterilized device has substantial challenges.

This disclosure describes a disposable ATF system and methods of use that may overcome one or more of these barriers to constructing and using a disposable ATF device suitable for intensified cell culture production.

### SUMMARY

In an aspect of the present disclosure, the bioreactor filtration system may comprise a hollow fiber filter module. The hollow fiber filter module may comprise a filter within a filter housing, the filter housing comprising a first end, a second end, and at least one permeate port, the hollow fiber filter module defining a feed/retentate channel and a permeate channel separated from the feed/retentate channel by the filter. The system may comprise first and second culture vessels attached to each of the first and second ends of the hollow fiber filter module, respectively. Each culture vessel may comprise an outer portion, an inner flexible vessel disposed within the outer portion, said inner flexible vessel configured to change in volume in response to a change in pressure in the outer portion, an outer port fluidly connected to the outer portion, and an inner port fluidly connected to the inner flexible vessel and fluidly isolated from the outer portion, wherein the feed/retentate channel may be in fluid communication with each inner port. The system may comprise a pressure source in fluid communication with each of the outer portions of the culture vessels. The first and second valves may be interposed between the pressure source and the first and second outer portions respectively.

In various embodiments, the first and second culture vessels may be disposed on first and second scales, respectively. The system may be gamma sterilizable. The system may be single use or multi use. The hollow fiber filter module may be single-use. One or both of the inner flexible vessels may be single-use. The hollow fiber filter module may be replaceable. The system may further comprise a cabinet. One or more of the hollow fiber filter module, the first and second culture vessels, and/or the pressure source may be installed in the cabinet. The system may further comprise a controller. The controller may be coupled to the pressure source. The controller may be coupled to a user interface.

In an aspect of the present disclosure, a filtration system may comprise a hollow fiber filter module. The hollow fiber filter module may comprise a first end, a second end, and at least one permeate port. The hollow fiber filter module may define a feed/retentate channel and a permeate channel. The permeate channel may be separated from the feed/retentate channel by the filter. The filtration system may comprise first and second fluid vessels attached to each of the first and second ends of the hollow fiber filter module, respectively. Each fluid vessel may comprise an outer portion. Each fluid vessel may comprise an inner flexible disposed within the outer portion and fluidly isolated from the outer portion. Each inner flexible vessel may be configured to translate a change in pressure in the outer portion to a retentate contained therein. An inner port may be fluidly connected to each respective inner flexible vessel. Each inner port may be configured to provide a flow therefrom in response to the change in pressure. Each feed/retentate channel may be in fluid communication with each inner port. A pressure source may be in fluid communication with each of the outer portions of the fluid vessels. The pressure source may be configured to effect the change in pressure.

In various aspects, the filtration system may further comprise a first fluid source coupled to the first fluid vessel. The filtration system may further comprise a second fluid source coupled to the second fluid vessel. The first fluid source, the second fluid source, or both may comprise a bioreactor.

In an aspect, a method of filtering bioreactor fluid may comprise alternating the flow of a fluid through a feed/retentate channel of a hollow fiber filter module between first and second culture vessels using a pressure source. Each culture vessel may comprise an outer portion, an inner flexible vessel disposed within the outer portion, said inner flexible configured to change in volume in response to a change in pressure in the outer portion, an outer port fluidly connected to the outer portion, and an inner port fluidly connected to the inner flexible vessel and fluidly isolated from the outer portion, wherein the feed/retentate channel is in fluid communication with each inner port. Fluid may flow through the hollow fiber filter module from a first inner flexible vessel to a second inner flexible vessel when pressure is introduced into a first outer portion surrounding the first inner flexible vessel. The system may alternate when pressure is introduced into a second outer portion surrounding the second inner flexible vessel.

In various embodiments, a resulting permeate may be removed from the system. The pressure system may comprise positive pressure or vacuum. The rate of flow may be determined by monitoring a change in weight of at least one of the first and second culture vessels over time. The fluid may be introduced into the system using batch or continuous processing. A permeate volume may be determined by monitoring a change in a combined weight of the first and second culture vessels over time.

In an aspect of the present disclosure, a method of filtering a bioreactor fluid may comprise creating a pressure differential between first and second vessels. The pressure differential may cause a responsive flow between third and fourth vessels. The third vessel may be disposed within the first vessel. The third vessel may be fluidly isolated from the first vessel. The fourth vessel may be disposed within the second vessel. The fourth vessel may be fluidly isolated from the second vessel. A hollow fiber filtration module may be fluidly connected between the third vessel and the fourth vessel.

In various aspects, the method of filtering a bioreactor fluid may further comprise alternating the pressure differential between the first and second vessels. The method may further comprise removing a permeate collected from the hollow fiber filtration module.

### BRIEF DESCRIPTION OF THE DRAWINGS

FIG. 1 is a schematic illustration of a filter system according to one or more embodiments of the present disclosure.

FIG. 2 illustrates an example of a communications architecture of the system 100 of FIG. 1.

FIG. 3 illustrates an example of a storage medium which may be implemented in the system 100 of FIG. 1.

FIG. 4 illustrates a computing platform of embodiments described herein.

### DETAILED DESCRIPTION

### Overview

Embodiments of the present disclosure relate generally to systems and methods for perfusion cell culture involving alternating fluid flows between first and second flexible vessels. Fluids such as suspension cell cultures pass through a tangential flow filtration apparatus as they move between the first and second vessels. As the fluids flow through the filter, they are separated into (I) a permeate flow comprising material that has passed through a membrane of the tangential flow filtration apparatus, and (II) a feed/retentate flow that has not passed through a membrane of the tangential flow filtration apparatus.

This disclosure describes a disposable ATF system suitable for supporting high density cell culture processes. This disclosure also provides methods for obtaining a high filtration performance in a sterile environment with the disposable ATF device. The present disclosure is based, at least in part, on the discovery that the use of vacuum pressure can reduce the shear stress on cell culture fluid even with increased flow. Further, no pressure sensors may be required on the flow paths to monitor the process, as this can be achieved by precise regulation of the vacuum sources. The device described within this disclosure may monitor the flow rates by placing the vessels of the device on scales. Further, the device may be single or multi-use, it may be used with cell cultures from batch or continuous processing, and it is gamma sterilizable.

Various embodiments may include preassembled and/or partially assembled combinations of components, which will be understood to allow for selective replacement of disposable components alongside maintenance of longer-lasting components, thereby improving sterility and/or sustainability of filter systems. Components may be housed, for example, in a cabinet or other structure.

Automated and/or user-based control of systems described herein may be enabled by communicative control of pressure systems, for example, via electronic instruction. In many embodiments, filter systems may be coupled to a controller and/or user interface enabling precise and/or simple regulation of flow, thereby improving reliability, ease of maintenance, and/or other aspect of use of filter systems.

Material is impelled between the flexible vessels by creating a pressure differential between them. Such a pressure differential may be created by any suitable means, including, without limitation, by gravity, by the application of positive pressure, and/or the application of negative pressure. In certain embodiments, the vessels possess sufficient flexibility to accommodate fluid flows without the need for dead space, i.e., the flexible vessels can collapse when emptied and expand to hold the full fluid volume in the system. In some cases, the flexible vessels comprise a flexible polymer such as silicone, latex, or like material suitable for sterilization by irradiation, gas exposure, or other sterilization means used in the art.

Positive pressure is applied in certain embodiments by direct mechanical compression of one or both vessels. This compression can be achieved manually, e.g., by squeezing the vessels, and/or mechanically, by compression, e.g., using a flexible bellows assembly or a piston driven compression system. In some embodiments, positive and/or negative pressure can be applied by placing the flexible vessel within a larger vessel and increasing or decreasing a pressure in the larger vessel, wherein the pressure will then tend to be equalized in the inner vessel.

In certain embodiments, material is impelled between the flexible vessels through a hollow fiber filter module. The hollow fiber filter module defines a feed/retentate channel and a permeate channel separated from the feed/retentate channel by a filter membrane such as a tangential flow filter element. When material is passed through the hollow fiber filter module, the material is separated into two streams: a permeate flows across the filter membrane, while a retentate passes into the vessel. The permeate may contain any number of species including without limitation a biological product e.g., monoclonal antibodies, recombinant proteins, microparticles, nanoparticles, vaccines, and/or viral vectors. Alternatively or additionally, the permeate may comprise waste, contaminant or other undesirable species. Accordingly, the permeate may be, variously, collected for further processing or discarded. Intact viable cells may remain in the retentate.

In some embodiments, the cell culture is the product. In some embodiments, the product is protein expressed by the cells, which is collected on the permeate.

In some embodiments, the vessels are comprised of an inner vessel and an outer vessel. The inner vessel is made of any flexible material such as multi-layer polyethylene (PE) film, or the like. The outer vessel is made of any flexible or inflexible material such as multi-layer PE film, silicone, or the like. The inner vessel is enclosed within the outer vessel. Pressure is applied to the outer vessel, which applies an equalizing pressure in the inner vessel. The system includes a series of ports or other similar connectors. Said ports connects an inner vessel with a hollow fiber filter module. Ports are used to fill and/or drain the inner vessel. Other ports are used to connect the outer vessel to a pressure source. The ports may be separate from other items of the system. Such ports may be sterilized.

In various embodiments, the material is placed into the flexible vessels before alternating the flow of fluid through the hollow fiber filter module. In some embodiments, the flexible vessels receive a continuous flow of material.

In some embodiments, a pressure source is connected via a port to an outer vessel. A pressure source can supply positive and/or negative pressure. If a single pressure source is used, an outer vessel may comprise a one-way valve in order to release excess pressure. In various embodiments, more than one pressure source can connected to the system. If more than one pressure source is used, each pressure source connects to an outer vessel.

The hollow fiber filter module may comprise a hollow fiber filter. Hollow fiber filters may be comprised of modified polyethersulfone, polysulfone, polyethersulfone, mixed cellulose ester, and the like. Examples of appropriate filters are described in U.S. Publication 2019/0276790, filed on Mar. 8, 2019 and published on Sep. 12, 2019, hereby incorporated by reference in its entirety.

In some embodiments, the filter and vessels are preassembled. In some embodiments, a flow path such as Proconnex is used to connect the filter and vessels.

In some embodiments, the filter and vessels are assembled as a system. In some embodiments, the filter and vessels are separate and may be assembled for use.

Reference is now made to the drawings, wherein like reference numerals are used to refer to like elements throughout. In the following description, for purposes of explanation, numerous specific details are set forth in order to provide a thorough understanding thereof. However, the novel embodiments can be practiced without these specific details. In other instances, well known structures and devices are shown in block diagram form to facilitate a description thereof. The intention is to cover all modifications, equivalents, and alternatives consistent with the claimed subject matter.

In the Figures and the accompanying description, the designations “a” and “b” and “c” (and similar designators) are intended to be variables representing any positive integer. Thus, for example, if an implementation sets a value for a=5, then a complete set of components 122 illustrated as components 122-1 through 122-a may include components 122-1, 122-2, 122-3, 122-4, and 122-5. Embodiments are not limited in this context.

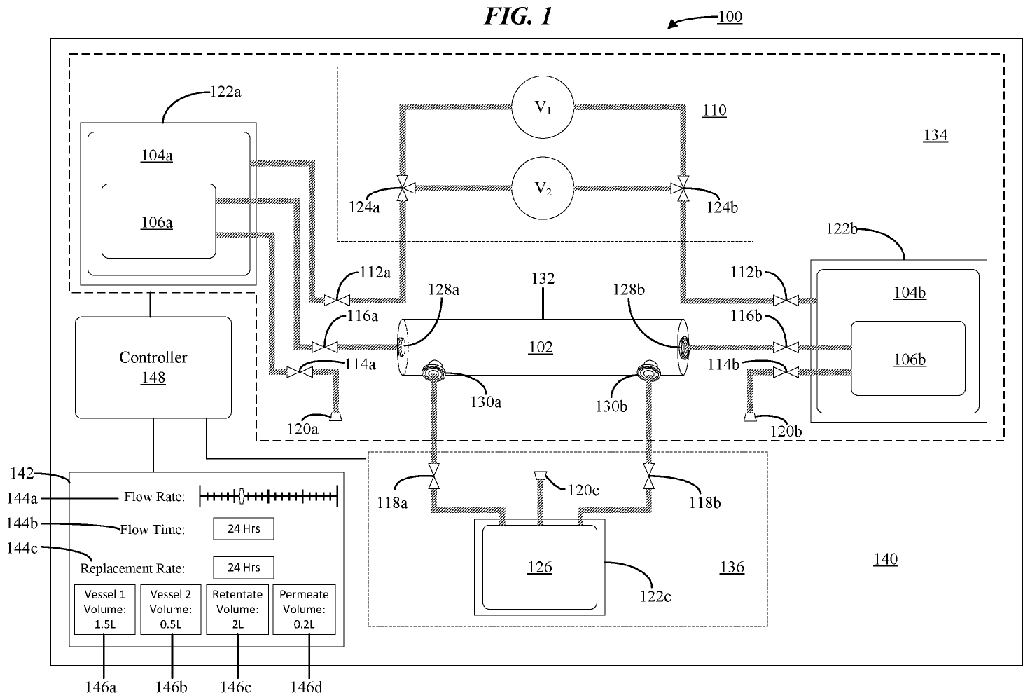

FIG. 1 depicts a system 100 according to various embodiments of the present disclosure. System 100 may be configured to filter a fluid (e.g., feed, cell culture fluid, etc.). System 100 (e.g., filtration system) includes a hollow fiber filter module 102 coupled to inner vessels 106a, b disposed within respective outer vessels 104a, b. By adjusting a pressure within one or both of outer vessels 104a, b, a pressure differential may be created between inner vessels 106a, b. The pressure differential may cause a flow from the higher-pressured inner vessel 106a or inner vessel 106b to the other, particularly through hollow fiber filter module 102. Hollow fiber filter module 102 may separate permeate from feed/retentate system 134 into permeate collection system 136. In various embodiments, feed/retentate system 134 and/or permeate collection system 136 may be installed in housing 140 and/or regulated via a controller 148 communicatively coupled to a user interface 142. Methods and elements described herein may enable high degrees of control over the flow, particularly resulting in lower shear stress being applied to flow contents than with conventional ATF systems. Without wishing to be bound to any theory, it is believed that presently disclosed embodiments modulating flow between inner vessels 106a, b via effecting a pressure change in one or both of outer vessels 104a, b may subject fluid to lower levels of shear stress, for example, by allowing pressure to be dispersed equally about at least one of inner vessels 106a, b such that pressure vectors on the fluid are distributed, resulting in lower shear stress to the fluid and gentler flow than in conventional systems.

Hollow fiber filter module 102 (e.g., hollow filter cartridge, hollow fiber filtration module, hollow fiber module, or the like) may include at least one hollow fiber filter. Such a filter is made as a cartridge that comprises multiple hollow fibers (HF) that run in parallel along the length of the cartridge and are embedded at each end of the cartridge (preferably with a potting agent); the lumens at the end of the HFs are retained open, thus forming a continuous passage through each of the lumens from one end of the cartridge to the other, i.e., from a cartridge entrance end, to a cartridge exit end. The hollow fibers are enclosed by the outer wall of the cartridge (i.e., the cartridge wall) and a potting layer at their ends. As a result, there is a chamber bounded by the cartridge wall and the outer walls of the HFs. That chamber can be used as the filtrate chamber. The intra-lumenal (e.g., internal, interstitial) spaces of the HFs are considered collectively to constitute part of the retentate chamber in systems presently disclosed.

The walls of the lumens of a hollow fiber filter are permeable, conveniently providing a barrier that is either fully permeable or selectively permeable. The selectively permeable hollow fiber walls may range in selectivity that ranges the entire gamut of membrane pore sizes, commonly classified as osmotic membranes, and from ultrafiltration microfiltration to macrofiltration and also micro-carrier filtration, where, for example,), the pore size range is about 10-500 kDa and 0.2-100 micron. Pore sizes of about 0.2 micron are commonly used for retaining cells and allowing metabolites and other molecules or molecular complexes to pass throughout the pores. On the other hand, ultrafiltration pore sizes in the range 10 kDa to 500 kDa, are preferred for retaining not only the cells, but molecules and molecular complexes, e.g., produced by the cells, that are larger than the pore sizes. Macrofiltration membranes range from 7 to 100 um and are used to retain microcarriers or larger cells.

The outer walls of filter cartridge, e.g., for use in the new disposable ATF pump units, are often non-permeable and commonly have ports from which filtrate can be drained and/or replaced. For purposes of some embodiments of the enclosed filtration systems, however, the filter cartridge can include an outer wall that constitutes a barrier that may be non-selective (fully permeable), but is preferably semi-permeable, (not allowing dissolved matter (e.g., molecules and molecular complexes) larger than the pore sizes in the barrier to pass through the barrier and not allowing particulate matter larger than the pore sizes to pass through the barrier). Pore sizes in the range 10 kDa to 500 kDa are preferred for retaining only molecules and molecular complexes larger than the pore sizes. However, the pore sizes can be made small enough or large enough, so that, respectively, the barrier is highly restrictive, allowing only small salts and their components to pass through or allowing molecules or particles larger than 500 kDa to pass through the membrane. Such membrane selectivity is not only restricted to pore size but to other membrane properties, including: charge, hydrophobicity, membrane configuration, membrane surface, pore polarity, etc.

Hollow fiber filter module 102 may be fluidly coupled via one or more ports to other elements of system 100. Particularly, ports 128a, b may fluidly couple hollow fiber filter module 102 to a feed/retentate system 134, and ports 132a, b may fluidly couple hollow fiber filter module 102 to a permeate collection system 136. Flow through one or more of ports 128a, b and/or of ports 130a, b may be regulated via respective valves 116a, 116b, 118a, and 118b, each of which may independently be controlled manually, automatically, or both.

Inner vessels 106a, b can be connected to the hollow fiber filter module 102 via fluid connections to respective ports 128a, b. In particular, inner vessels 106a, b may be configured to allow retentate flow between each other, and therefore through, hollow fiber filter module 102. In many embodiments, inner vessels 106a, b may be formed of sterilizable, flexible and/or elastic material which may translate externally applied pressure to a fluid volume contained therein. Inner vessels 106a, b may be made of materials non-toxic to cell culture fluids, and inner vessels 106a, b may be impermeable to fluid flow. In various embodiments, inner vessels 106a, b may be cell culture vessels or the like.

The inner vessels 106a, b may be contained within respective outer vessels 104a, b, which may be used to affect external pressure applied to inner vessels 106a, b. Outer vessels 104a, b may be formed of rigid material, such as metal and/or an inflexible polymer capable of withstanding internally applied pressure. Outer vessels 104a, b may contain fluid, which in many cases, may be entirely separated from and unexposed to the content of inner vessels 106a, b. Outer vessels 104a, b may be fluid-tight with an exception to connection to a pressure source 110. Accordingly, a control of fluid volume within outer vessels 104a, b may generate a vacuum and/or pressure application to inner vessels 106a, b. As inner vessels 106a, b are flexible, pressure differentials created between outer vessels 104a, b may thus generate corresponding pressure differentials between inner vessels 106a, b, resulting in a responsive fluid flow between inner vessels 106a, b towards an equalization of pressure.

Outer vessels 104a, b can be connected to pressure source 110 (e.g., pump). One or more outer vessels 104a, b can connect to one or more pressure sources 110 (connection to multiple pressure sources 110 not shown for the sake of simplicity in the drawings), each of which may include one or more pumps (e.g., V₁, V₂ may be two pumps set to work in coordination with each other, thereby reducing load on each). Pressure source 110 may use a natural and/or artificial force to apply pressure to a fluid (e.g., gravity, diaphragm pump, air flow pump, etc.). Pressure source 110 may include one or more valves 124a, b which regulate flow to and/or from components of pressure source 110. Pressure source 110 may generate and/or comprise a positive pressure, a vacuum, or both (e.g., an alternating pressure). The outer vessels 104a, b may connect to pressure source 110 via respective connection valves 112a, b. Valves 124a, b, valves 112a, b, or any combination thereof may be used to regulate flow to and/or from pressure source 110, and in various embodiments may comprise or be ports establishing a fluid pathway therethrough.

It will be recognized that alternating a pressure application on outer vessels 104a, b via pressure source 110 may generate a responsive fluid flow between inner vessels 106a, b, particularly through hollow fiber filter module 102. Without wishing to be bound by any theory, it is believed that the indirect application of pressure to cause a fluid flow may enable system 100 to provide a gentler and/or lower shear pressure to the fluid/feed/retentate.

The inner vessels 106a, b can be filled using respective fill/drain ports 120a, b. In various embodiments, ports 120a, b may be fluidly coupled to at least one bioreactor (not shown).

Alternatively, or additionally, it is currently contemplated that inner vessels 106a, b may be filled with cell culture fluid and/or seeded with cells via flow through ports 120a, b. Cells may be cultured within inner vessels 106a, b, for example, prior to and/or during filtration through hollow fiber filter module 102. Accordingly, one or both of inner vessels 106a, b may function as a bioreactor.

Upon application of pressure source 100 to fluid in one or both of outer vessels 104a, b, fluid (feed/retentate) within the inner vessels 106a, b may flow through respective connection valves 116a, b through the hollow fiber filter module 102, wherein the fluid may pass through a feed/retentate and/or permeate channel.

In particular, permeate from hollow fiber filter module 102 may be collected in vessel 126. Once the fluid has passed through the permeate channel (e.g., into hollow fiber filter module 102), it may be removed from the system 100 (e.g., via drain port 120c).

One or both of feed/retentate system 134 and/or permeate collection system 136 may be installed in housing 140 (e.g., cabinet or other unit). Housing 140 may be sterilizable. One or more elements of feed/retentate system 134 and/or permeate collection system 136 may be removable and/or otherwise replaceable from housing 140. For example, inner vessels 106a, b, outer vessels 104a, b, vessel 126, hollow fiber filter module 102, or any combination thereof may be replaced independently or in conjunction with other components. For example, inner vessels 106a, b, outer vessels 104a, b, vessel 126, hollow fiber filter module 102, or any combination thereof may be independently, or in conjunction with other components, reusable (multi-use), manufactured for limited use, or single use. Replacements may be the same or different sizes as original components. For example, housing 140 may support installation of various sizes of hollow fiber filter modules 102 such that a longer hollow fiber filter module 102 may be used as a cell culture volume increases (e.g., if system 100 is used for perfusion in a seed train cell culture system, the same system 100 or a similar system 100 may be connected to progressively larger bioreactors, wherein the system 100 coupled to the larger of the bioreactors comprises a hollow fiber filter module 102 with a greater length than that of the smaller of the bioreactors (not illustrated)).

It will be understood that housing 140 may include any number of drawers, latches, clamps, and/or other features useful for securing elements of feed/retentate system 134 and/or permeate collection system 136 (not shown for the sake of simplicity in the drawings). Housing 140 may enable one or more elements of system 100 to be efficiently packaged and/or managed to and/or by users, improving a simplicity of use over many conventional filter systems. In many embodiments, various elements of system 100 may be presterilized and packaged in housing 140 so that a method of preparation of system 100 involves only filling one or both inner vessels 106a, b with fluid to be filtered. In various embodiments, a method of preparation of system 100 may involve only coupling system 100 to a power source (not shown) and filling one or both inner vessels 106a, b with fluid to be filtered.

According to various embodiments described herein, one or more scales 122a-c may be used to monitor filtering processes. In particular, outer vessel 104a may be on and/or otherwise coupled to scale 122a, outer vessel 104b may be on and/or otherwise coupled to scale 122b, and/or vessel 126 may be on and/or otherwise coupled to scale 122c.

Any combination of scales 122a-c may be used to measure changes in weight within and/or between outer vessels 104a, b (along with respective inner vessels 106a, b), vessel 126, or any combination thereof. For example, a first weight may be measured as the sum of weights detected by scales 122a, b. In the same example, pressure source 110 may cause an increase of fluid (and therefore of pressure) within outer vessel 104a, which may result in a corresponding fluid flow from inner vessel 106a across hollow fiber filter module 102. Retentate from the flow may continue into inner vessel 106b. A corresponding volume of fluid may flow out of outer vessel 104b. However, permeate from hollow fiber filter module 102 may exit one or both of ports 130a, b and enter vessel 126. Accordingly, in the same example, scale 122c may detect an increased weight corresponding to a decrease in summed weight detected by scales 122a, b. Based on known standards and/or calculations of density and/or weight of feed/retentate and permeate, volume of flow through elements of system 100 may be estimated, for example, without a need for pressure sensors, which may be expensive.

Operations of pressure source 100 may be adjusted based on calculations/estimations of flow using measurements of scales 122a-c. In some embodiments, flow through one or more of ports 120a-c may be regulated based on measurements from scales 122a-c. For example, an increase in volume of permeate calculated based on a measurement of scale 122c may reach a threshold value, at which point feed/retentate may be replenished through one or both of ports 120a, b, permeate may be drained from system 100 via port 122b, or any combination thereof.

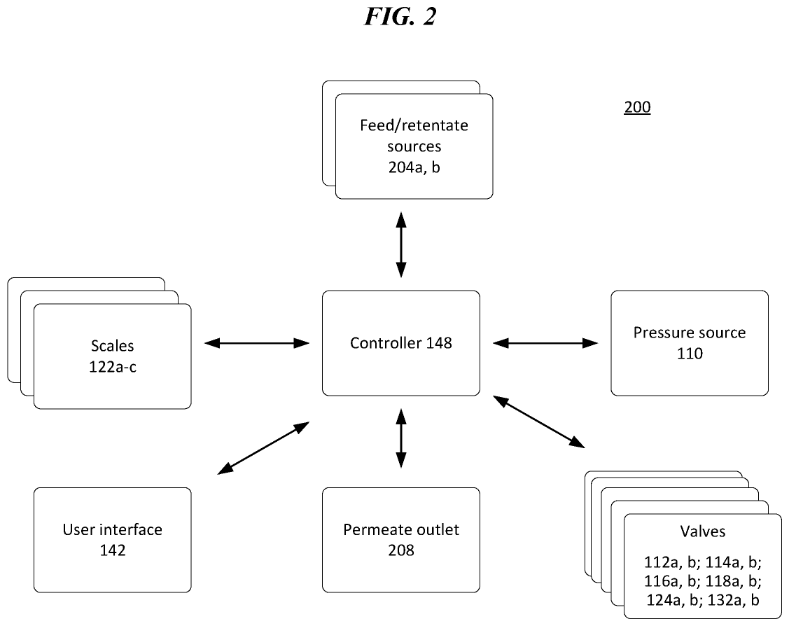

In various embodiments, one or more of feed/retentate system 134 or permeate collection system 136 may be managed and/or monitored using a controller 148. Controller 148 may be communicatively coupled to one or both of feed/retentate system 134 and/or permeate collection system 136. Controller 148 may be communicatively coupled to elements of system 100 via environment 200 as described with respect to FIG. 2. Controller 148 may be permanently and/or removably installed in housing 140, and in many cases, may include a sterilizable covering (not shown).

Controller 148 may be coupled to a user interface 142, which may be useful for managing one or both of feed/retentate system 134 or permeate collection system 136. In many embodiments, user interface 142 may be displayed on a screen or monitor installed on and/or in housing 140.

User interface 142 may include one or more controls 144a-c useful for inputting instructions for managing operations of feed/retentate system 134 and/or permeate collection system 136. For example, as illustrated, pump speed of pressure source 110 may be changed via control 144a, affecting a resulting fluid flow through hollow fiber filter module 102. Control 144b may direct a duration of operation of one or more aspects of system 100. Control 144c may coordinate flows through ports 120a-c so as to manage a replacement rate of cell culture fluid (e.g., to maintain a desired total volume of system 100).

Additionally, or alternatively, various data panels 146a-d may display current and/or periodic data measured from system 100 (e.g., measurements of scales 122a-c, estimations of volumes within at least one of outer vessels 104a, b, inner vessels 106a, b, and/or vessel 126.

It will be understood that user interface 142 may include various input methods for instructions, including but not limited to slide bars, text entry, buttons, dials, or the like. Additionally, or alternatively, user interface 142 may display other information than that described above, which may be useful for managing and/or monitoring elements of system 100. For example, a timestamp and/or other experimental data may be displayed.

It will be readily appreciated by those of skill in the art that various embodiments described herein may present one or more improvements over conventional systems in increasing control, automation, scalability, production or economy. Embodiments described herein may have one or more improvements over conventional systems in decreasing a footprint, shear stress, cost, time requirement, or other constraint associated with conventional system(s). Various embodiments may be used in batch and/or continuous processing applications. For example, embodiments may be used in fed-batch cell culture perfusion applications by using one or multiple systems 100 as described herein.

In an example of a seed train optimization of cell cultures, system 100 may be used for perfusion purposes. While a seed train cell culture volume is sufficiently small (e.g., the same or less than a combined volume of inner vessels 106a, b), cell culture may be inoculated directly in one or both of inner vessels 106a, b such that system 100 functions as a bioreactor. Nutrients and/or cell culture medium may be added via ports 120a, b. As cell culture volume increases, for example, past a threshold value, pressure source 100 may be used to remove permeate through hollow fiber filter module 102.

In various examples, when a cell density and/or viability threshold is reached, inner vessels 106a, b, hollow fiber filter module 102, or both may be replaced with respectively larger-volume vessels 106a, b, or a higher capacity hollow fiber filter module 102. Alternatively, cell culture may be moved to a second system 100 comprising inner vessels 106a, b with larger volumes.

When the seed train has reached a stage beyond a capacity of inner vessels 106a, b to function as stand-alone bioreactors, cell culture may be transferred to a larger bioreactor system and inner vessels 106a, b may be directly and fluidly coupled to the same via ports 120a, b. System 100 may be used for perfusion of the cell culture, where cell culture is processed through inner vessels 106a, b, hollow fiber filter module 102, and vessel 126 in coordination with the growth of the cell culture in the bioreactor (not illustrated). Coordination may be managed, in many embodiments, via controller 148. Several systems 100 may be used in series with bioreactors of varying volumetric capacities.

### Priming of Filters for Flow Systems

Selectively permeable hollow fibers as discussed herein must be wet with a liquid compatible with the fluid substance be filtered. For example, in cell culture the membrane must be wet with water-based solutions that are compatible with cell culture growth. Many membranes require alcohol containing solutions to initially wet the pores and achieve flux rates during operation that are needed to perform the filtration process. FIG. 1 shows the ports and fluid bags that can be used to add fluid to the ATF device (e.g., ports 120a-c, vessels 106a, b, and/or vessel 126). Flushing with serum free media in a sterile environment can then be performed using the alternating pumping action of the ATF device (e.g., prior to filling vessels 106a, b with cell culture material). Then the flush fluid can be drained from the port and the device is ready to operate in the cell culture process while maintaining a sterile environment. In various embodiments, hollow fiber filter module 102 may be pre-wet before installation into system 100 of FIG. 1.

### Systems and Structures for Controlling Flow Systems

FIG. 2 illustrates an example of a communications environment 200 of system 100, as described with respect to FIG. 1. In particular, a controller 148 may be communicatively coupled with one or more of user interface 142, scales 122a-c and/or pressure source 110 as described with respect to FIG. 1, in addition to retentate sources 204a, b, permeate outlet 208, or any combination thereof. In many embodiments, retentate sources 204a, b may include or be otherwise coupled to ports 120a, b, and/or permeate outlet 208 may include or be otherwise coupled to port 120c as described to FIG. 1. Communications as described with respect to FIG. 2 may be wired, via a wireless network, or any combination thereof.

Controller 148 may communicate with one or more of the illustrated elements alone or in coordination in order to regulate flow through an ATF as described herein. For example, scales 122a-c may communicate weights of retentate and/or permeate volume to controller 148 periodically or in real time. In many embodiments, as a permeate volume weight increases, controller 148 may direct retentate sources 204a, b to replenish a retentate supply into one or both of vessels 106a, b as described with respect to FIG. 1, particularly through respective ports 120a, b.

In various embodiments, controller 148 may direct permeate outlet 208 to open and/or to release permeate from system 100 based on an increased report of permeate weight and/or of decreased retentate weight received from scales 122a-c. Controller 148 may direct permeate outlet 208 and one or more of retentate sources 204a, b in coordination with each other in order to maintain a substantially constant total fluid volume of system 100.

Controller 148 may direct operations of pressure source 110, for example, to alternate pressure increases and decreases between outer vessels 104a, b as described with respect to FIG. 1. Controller 148 may be configured to, for example, send instructions to pressure source 110 to determine a rate and/or magnitude of pressure changes in one of both of outer vessels 104a, b. In many embodiments, controller 148 may send instructions to pressure source 110 based on determinations of relative volumes in one or more of inner vessels 106a, b (e.g., based on reports of weight from scales 122a-c). Additionally, or alternatively, controller 148 may send instructions to pressure source 110 based on determinations of a permeate volume (e.g., based on reports of weight from scales 122a-c).

Additionally, or alternatively, controller 148 may be individually or collectively coupled to any combination of valves 112a, b, 114a, b, 116a, b, 118a, b, 124a, b, and/or 132a, b. In various embodiments, controller 148 may direct an operation of a valve 112a, b, 114a, b, 116a, b, 118a, b, 124a, b, and/or 132a, b to increase and/or decrease flow through a respective flow path. Accordingly, flow through any part of system 100 may be regulated via controller 148. In some embodiments, any or all of valves 112a, b, 114a, b, 116a, b, 118a, b, 124a, b, and/or 132a, b may be manually controlled (e.g., without the use of controller 148).

In various embodiments, operations of controller 148 as described above may be automated. In some embodiments, one or more above-described operations of controller 148 may be based on receiving an instruction via user interface 142. For example, controller 148 may direct pressure source 110 to adjust pressures of vessels 104a, b at a particular rate and/or for a particular duration of time based on instructions received via respective controls 144a, b, as described with respect to FIG. 1. In the same or in another example, controller 148 may direct pressure source 110 to allow flow through one or more of retentate sources 204a, b, and/or permeate outlet 208 based on instructions to replenish retentate as received through control 144c.

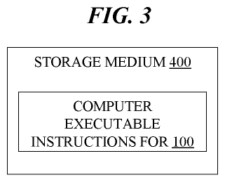

FIG. 3 illustrates an example of a storage medium 400 to store processor data structures, particularly for controlling aspects of system 100 as described with respect to FIG. 1. In many embodiments, controller 148 as described with respect to FIGS. 1 and 2 may include a storage medium 400. Storage medium 400 may comprise an article of manufacture. In some examples, storage medium 400 may include any non-transitory computer readable medium or machine-readable medium, such as an optical, magnetic or semiconductor storage. Storage medium 400 may store diverse types of computer executable instructions, such as instructions to implement logic flows and/or techniques described herein. Examples of a computer readable or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions may include any suitable type of code, such as source code, compiled code, interpreted code, executable code, static code, dynamic code, object-oriented code, visual code, and the like.

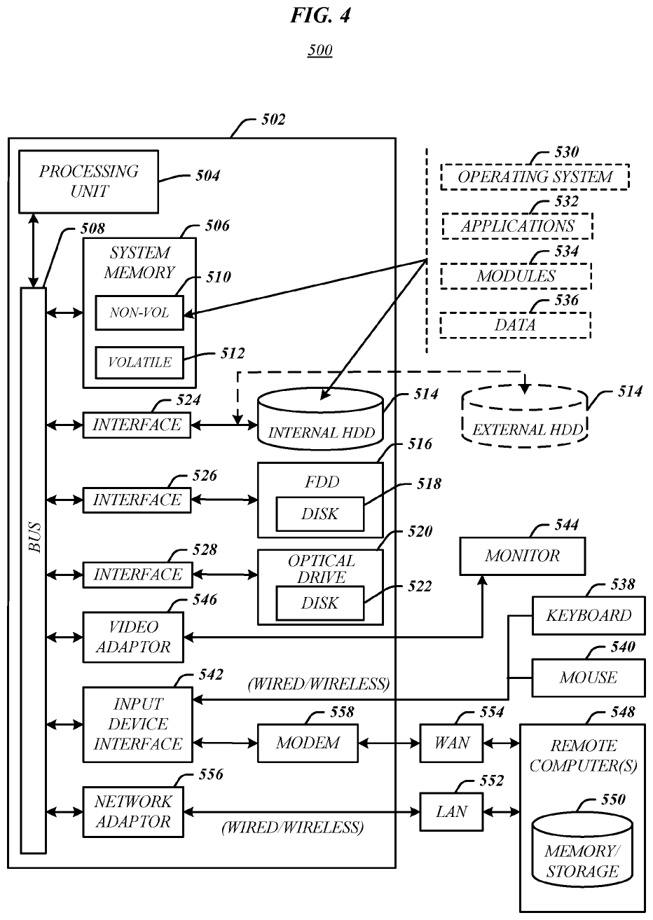

FIG. 4 illustrates an embodiment of an exemplary computing architecture 500 that may be suitable for implementing various embodiments as previously described such as controller 148, described with respect to FIG. 1 and/or FIG. 2. In various embodiments, the computing architecture 500 may comprise or be implemented as part of an electronic device. In some embodiments, the computing architecture 500 may be representative, for example, of one or more component described herein. In some embodiments, computing architecture 500 may be representative, for example, of a computing device that implements or utilizes one or more of user interface 142, and/or one or more techniques described herein. Embodiments are not limited in this context.

A computer-related “system” and “component” and “module” may be intended to refer to a computer-related entity, either hardware, a combination of hardware and software, software, or software in execution, examples of which are provided by the exemplary computing architecture 500. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

The computing architecture 500 includes various common computing elements, such as one or more processors, multi-core processors, co-processors, memory units, chipsets, controllers, peripherals, interfaces, oscillators, timing devices, video cards, audio cards, multimedia input/output (I/O) components, power supplies, and so forth. The embodiments, however, are not limited to implementation by the computing architecture 500.

As shown in FIG. 5, the computing architecture 500 comprises a processing unit 504, a system memory 506 and a chipset and bus 508. The processing unit 504 can be any of various commercially available processors. Dual microprocessors, multi-core processors, and other multi processor architectures may also be employed as the processing unit 504.

The chipset and bus 508 provides an interface for system components including, but not limited to, the system memory 506 to the processing unit 504. The chipset and bus 508 can include any of several types of bus structure that may further interconnect to a memory bus (with or without a memory controller), a peripheral bus, and a local bus using any of a variety of commercially available bus architectures. Interface adapters may connect to the chipset and bus 508 via a slot architecture. Example slot architectures may include without limitation Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and the like.

The system memory 506 may include various types of computer-readable storage media such as non-transitory computer-readable storage media in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory (e.g., one or more flash arrays), polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In the illustrated embodiment shown in FIG. 5, the system memory 506 can include non-volatile memory 510 and/or volatile memory 512. In some embodiments, system memory 506 may include main memory. A basic input/output system (BIOS) can be stored in the non-volatile memory 510.

In various embodiments, a computer 502 may be a controller 148 as described above. The computer 502 may include various types of computer-readable storage media in the form of one or more lower speed memory units, including an internal (or external) hard disk drive (HDD) 514, a magnetic floppy disk drive (FDD) 516 to read from or write to a removable magnetic disk 518, and an optical disk drive 520 to read from or write to a removable optical disk 522 (e.g., a CD-ROM or DVD). The HDD 514, FDD 516 and optical disk drive 520 can be connected to the chipset and bus 508 by an HDD interface 524, an FDD interface 526 and an optical drive interface 528, respectively. The HDD interface 524 for external drive implementations can include at least one or both of Universal Serial Bus (USB) and Institute of Electrical and Electronics Engineers (IEEE) 694 interface technologies. In various embodiments, these types of memory may not be included in main memory or system memory.

The drives and associated computer-readable media provide volatile and/or nonvolatile storage of data, data structures, computer-executable instructions, and so forth. For example, a number of program modules can be stored in the drives and memory units 510, 512, including an operating system 530, one or more application programs 532, other program modules 534, and program data 536. In one embodiment, the one or more application programs 532, other program modules 534, and program data 536 can include or implement, for example, the various techniques, applications, and/or components described herein.

A user can enter commands and information into the computer 502 through one or more wire/wireless input devices, for example, a keyboard 538 and a pointing device, such as a mouse 540. Other input devices may include microphones, infra-red (IR) remote controls, radio-frequency (RF) remote controls, game pads, stylus pens, card readers, dongles, finger print readers, gloves, graphics tablets, joysticks, keyboards, retina readers, touch screens (e.g., capacitive, resistive, etc.), trackballs, trackpads, sensors, styluses, and the like. These and other input devices are often connected to the processing unit 504 through an input device interface 542 that is coupled to the chipset and bus 508, but can be connected by other interfaces such as a parallel port, IEEE 994 serial port, a game port, a USB port, an IR interface, and so forth.

A monitor 544 or other type of display device is also connected to the chipset and bus 508 via an interface, such as a video adaptor 546 or other display driver. The monitor 544 may be internal or external to the computer 502. In many embodiments, a monitor 544 may display user interface 142, as described with respect to FIG. 1. In addition to the monitor 544, a computer typically includes other peripheral output devices, such as speakers, printers, and so forth.

The computer 502 may operate in a networked environment using logical connections via wire and/or wireless communications to one or more remote computers, such as a remote computer 548. In various embodiments, one or more migrations may occur via the networked environment. The remote computer 548 can be a workstation, a server computer, a router, a personal computer, portable computer, microprocessor-based entertainment appliance, a peer device or other common network node, and typically includes many or all the elements described relative to the computer 502, although, for purposes of brevity, only a memory/storage device 550 is illustrated. The logical connections depicted include wire/wireless connectivity to a local area network (LAN) 552 and/or larger networks, for example, a wide area network (WAN) 554. Such LAN and WAN networking environments are commonplace in offices and companies, and facilitate enterprise-wide computer networks, such as intranets, all of which may connect to a global communications network, for example, the Internet.

When used in a LAN networking environment, the computer 502 is connected to the LAN 552 through a wire and/or wireless communication network interface or adaptor 556. The adaptor 556 can facilitate wire and/or wireless communications to the LAN 552, which may also include a wireless access point disposed thereon for communicating with the wireless functionality of the adaptor 556.

When used in a WAN networking environment, the computer 502 can include a modem 558, or is connected to a communications server on the WAN 554, or has other means for establishing communications over the WAN 554, such as by way of the Internet. The modem 558, which can be internal or external and a wire and/or wireless device, connects to the chipset and bus 508 via the input device interface 542. In a networked environment, program modules depicted relative to the computer 502, or portions thereof, can be stored in the remote memory/storage device 550. It will be appreciated that the network connections shown are exemplary and other means of establishing a communications link between the computers can be used.

The computer 502 may be operable to communicate with wire and wireless devices or entities using the IEEE 802 family of standards, such as wireless devices operatively disposed in wireless communication (e.g., IEEE 802.16 over-the-air modulation techniques). This includes at least Wi-Fi (or Wireless Fidelity), WiMax, and Bluetooth™ wireless technologies, among others. Thus, the communication can be a predefined structure as with a conventional network or simply an ad hoc communication between at least two devices. Wi-Fi networks use radio technologies called IEEE 802.11x (a, b, g, n, etc.) to provide secure, reliable, fast wireless connectivity. A Wi-Fi network can be used to connect computers to each other, to the Internet, and to wire networks (which use IEEE 802.3-related media and functions).

### CONCLUSION

The foregoing disclosure has presented exemplary embodiments of filtration systems according to the present disclosure. These embodiments are not intended to be limiting, and it will be readily appreciated by those of skill in the art that various additions or modifications may be made to the systems and methods described above without departing from the spirit and scope of the disclosure.

It will be additionally, or alternatively, readily appreciated by those of skill in the art that various reductions to may be made to the systems and methods described above without departing from the spirit and scope of the disclosure. For example, various embodiments may include feed/retentate system 134, permeate collection system 136, and pressure source 110 but not include a controller 148, user interface 142, housing 140, or any combination thereof, as described with respect to FIG. 1.

Additionally, while the foregoing disclosure has focused primarily on hollow fiber filtration systems and their applications, it will be appreciated by those of skill in the art that the principles of the disclosure are applicable to other systems including conventional TFF, TFDF, and ATF systems.

1. A bioreactor filtration system, comprising:
a hollow fiber filter module comprising a filter within a filter housing, the filter housing comprising a first end, a second end, and at least one permeate port, the hollow fiber filter module defining a feed/retentate channel and a permeate channel separated from the feed/retentate channel by the filter;
first and second culture vessels attached to each of the first and second ends of the hollow fiber filter module, respectively, wherein each culture vessel comprises an outer portion, an inner flexible vessel disposed within the outer portion, said inner flexible vessel configured to change in volume in response to a change in pressure in the outer portion, an outer port fluidly connected to the outer portion, and an inner port fluidly connected to the inner flexible vessel and fluidly isolated from the outer portion, wherein the feed/retentate channel is in fluid communication with each inner port;
a pressure source in fluid communication with each of the outer portions of the culture vessels; and
first and second valves interposed between the pressure source and the first and second outer portions respectively.

2. The system of claim 1, wherein the first and second culture vessels are disposed on first and second scales, respectively.

3. The system of claim 1, wherein the system is gamma sterilizable.

4. The system of claim 1, wherein the hollow fiber filter module is single-use.

5. The system of claim 1, wherein one or both of the inner flexible vessels are single use.

6. The system of claim 1, wherein the system is multi-use.

7. The system of claim 1, wherein the hollow fiber filter module is replaceable.

8. The system of claim 1, wherein the system further comprises a cabinet in which the hollow fiber filter module, the first and second culture vessels, and the pressure source are installed.

9. The system of claim 1, wherein the system further comprises a controller coupled to the pressure source.

10. The system of claim 9, wherein the controller is coupled to a user interface.

11. A filtration system, comprising:
a hollow fiber filter module comprising a filter within a filter housing, the filter housing comprising a first end, a second end, and at least one permeate port, the hollow fiber filter module defining a feed/retentate channel and a permeate channel separated from the feed/retentate channel by the filter;
first and second fluid vessels attached to each of the first and second ends of the hollow fiber filter module, respectively, wherein each fluid vessel comprises an outer portion, an inner flexible vessel disposed within the outer portion and fluidly isolated from the outer portion, said inner flexible vessel configured to translate a change in pressure in the outer portion to a retentate contained therein, an inner port fluidly connected to the inner flexible vessel and configured to provide a flow therefrom in response to the change in pressure, wherein the feed/retentate channel is in fluid communication with each inner port; and
a pressure source in fluid communication with each of the outer portions of the culture vessels, the pressure source configured to effect the change in pressure.

12. The system of claim 11, further comprising a first fluid source coupled to the first fluid vessel, a second fluid source coupled to the second fluid vessel, or both.

13. The system of claim 12, wherein the first fluid source, the second fluid source, or both, comprises a bioreactor.

14. A method of filtering bioreactor fluid, comprising:
alternating the flow of a fluid through a feed/retentate channel of a hollow fiber filter module between first and second culture vessels using a pressure source,
wherein each culture vessel comprises an outer portion, an inner flexible vessel disposed within the outer portion, said inner flexible vessel configured to change in volume in response to a change in pressure in the outer portion, an outer port fluidly connected to the outer portion, and an inner port fluidly connected to the inner flexible vessel and fluidly isolated from the outer portion, wherein the feed/retentate channel is in fluid communication with each inner port,
wherein fluid flows through the hollow fiber filter module from a first inner flexible vessel to a second inner flexible vessel when pressure is introduced into a first outer portion surrounding the first inner flexible vessel, and
wherein the system alternates when pressure is introduced into a second outer portion surrounding the second inner flexible vessel.

15. The method of claim 14, wherein a resulting permeate is removed from the system.

16. The method of claim 15, wherein the pressure system comprises positive pressure or vacuum.

17. The method of claim 14, wherein the rate of flow is determined by monitoring a change in weight of at least one of the first and second culture vessels over time.

18. The method of claim 14, wherein the fluid is introduced into the system using batch or continuous processing.

19. The method of claim 14, wherein a permeate volume is determined by monitoring a change in a combined weight of the first and second culture vessels over time.

20-21. (canceled)
